(19) United States
(12) Patent Application Publication
(10) Pub. No.: US 2022/0178917 A1
(43) Pub. Date: Jun. 9, 2022

(54) BIOMOLECULAR SENSORS WITH DESALTING MODULE AND RELATED METHODS

(71) Applicant: FEMTODX, Beverly Hills, CA (US)

(72) Inventor: Pritiraj Mohanty, Beverly Hills, CA (US)

(73) Assignee: FemtoDx, Inc., Beverly Hills, CA (US)

(21) Appl. No.: 17/438,000

(22) PCT Filed: Mar. 13, 2020
(86) PCT No.: PCT/US2020/022544

§ 371 Date: Sep. 10, 2021

Related U.S. Application Data
(60) Provisional application No. 62/817,821, filed on Mar. 13, 2019.
(60) Provisional application No. 62/817,825, filed on Mar. 13, 2019.

(51) Int. Cl. G01N 33/543; G01N 1/34; B01L 3/00; G01N 27/12

(52) U.S. Cl. CPC G01N 33/5438; G01N 1/34; B01L 2300/0645; G01N 27/125; B01L 3/502715

(57) ABSTRACT

Systems and methods for removing ions from a sample (i.e., desalting) are generally described. In some embodiments, “desalting” comprises removing ions from a sample, the sample also comprising an analyte, such as a protein, a hormone, or an antigen. Unwanted ions can increase the noise when detecting or sensing a signal from an analyte within the sample.

### RELATED APPLICATIONS

This application claims priority under 35 U.S.C. § 119(e) to U.S. Provisional Application No. 62/817,821, filed Mar. 13, 2019, and entitled “BIOMOLECULAR SENSORS WITH INTEGRATED ELECTROPHORETIC DESALTING MODULE,” and U.S. Provisional Application No. 62/817,825, filed Mar. 13, 2019, and entitled “BIOMOLECULAR SENSORS WITH INTEGRATED MICRORESIN DESALTING MODULE,” which are incorporated herein by reference in its entirety for all purposes.

### TECHNICAL FIELD

Systems and methods for removing ions from a sample (i.e., desalting) are generally described.

### BACKGROUND

Biological samples may contain unwanted ions that may affect the detection of a species of interest (e.g., an analyte). Thus it can be useful to remove these ions or “desalt” the sample. Various methods can be used for such external desalting. Some common examples are centrifugal and gravitational desalting. In these desalting processes, an ion-rich fluid can be forced through a special resin. The resin can contain both large holes and smaller, winding tunnels. The salt ions may get trapped in the winding tunnels and therefore proceed more slowly through the resin. The larger analyte molecules may be too big to go into the tunnels, and therefore proceed through the larger holes, exiting the resin more quickly. The force to push the fluid through the resin is provided by gravity or centrifugally. However, gravitational forcing can be slow, and can also require the user to align the device along the force of gravity. Centrifugal forcing can be faster and more rapid, but requires a large centrifuge. For a sample that is to be tested with a biosensor for analyte presence, these challenges limit the possibility for the sample extraction and test to be performed by the end user. Therefore, improved systems and methods are needed.

### SUMMARY

Systems and methods for desalting a sample are generally described. In some embodiments, “desalting” comprises removing ions from a sample, the sample also comprising an analyte, such as a protein, a hormone, or an antigen. Unwanted ions can increase the noise when detecting or sensing a signal from an analyte within the sample. Thus, desalting may remove these unwanted ions (e.g., salts) in order to decrease the noise of the sample and provide a clearer signal when the analyte is sensed by a sensor. The subject matter of the present invention involves, in some cases, interrelated products, alternative solutions to a particular problem, and/or a plurality of different uses of one or more systems and/or articles.

In one aspect, a system for removing a plurality of ions from a sample, the system comprising a first electrode; a first porous material adjacent to at least a portion of the first electrode; a second electrode in electrical communication with the first electrode; and a second porous material adjacent to at least a portion of the second electrode.

In another aspect, a system for removing a plurality of ions from a sample, the system comprising a substrate; a desalting chamber proximate the substrate, where the desalting chamber comprises a first electrode, a first porous material adjacent to at least a portion of the first electrode, a second electrode in electrical communication with the first electrode, and a second porous material adjacent to at least a portion of the second electrode; a microfluidic channel; and a sensing chamber proximate the substrate, where the sensing chamber comprises at least one sensor, wherein the desalting chamber and the sensing chamber are in fluidic communication via the microfluidic channel.

In another aspect, a method of removing a plurality of ions from a sample is described, the method comprising flowing the sample into a desalting chamber where the desalting chamber comprises a first electrode, a first porous material adjacent to at least a portion of the first electrode, a second electrode in electrical communication with the first electrode, and a second porous material adjacent to at least a portion of the second electrode; applying a first voltage of a first sign to the first electrode; applying a second voltage of a second sign to the second electrode; attracting at least a portion of the plurality of ions towards the first electrode and the second electrode; flowing the sample into a microfluidic channel; flowing the sample into a sensing chamber, wherein the sensing chamber and the desalting chamber are fluidically connected via the microfluidic channel; and sensing an analyte within the sample.

In a different aspect, a system for removing a plurality of ions from a sample is described. The system comprises a microfluidic channel, where the microfluidic channel comprises a fluid inlet and a fluid outlet downstream the fluid inlet, wherein a valve is adjacent the fluid inlet; a piston disposed within the fluidic channel and proximate the fluid inlet; a force generator adjacent to the piston; and a porous material within the fluidic channel, wherein the porous material is disposed between the valve the fluid outlet, and wherein the piston is configured to move a sample downstream the microfluidic channel.

In yet another aspect, a system for removing a plurality of ions from a sample is described. The system comprises a substrate; a desalting chamber proximate the substrate where the desalting chamber comprises a microfluidic channel and the microfluidic channel comprises a fluid inlet and a fluid outlet downstream the fluid inlet, wherein a valve is adjacent the fluid inlet; a piston disposed within the fluidic channel and proximate the fluid inlet; a force generator adjacent to the piston; and a porous material within the fluidic channel, wherein the porous material is disposed between the valve the fluid outlet; and a sensing chamber proximate the substrate where the sensing chamber comprises at least one sensor, wherein the piston is configured to move a sample downstream within the microfluidic channel, and wherein the desalting chamber and the sensing chamber are in fluidic communication via the microfluidic channel.

In yet a different aspect, a method of removing a plurality of ion from a sample is described, the method comprising flowing a sample into a desalting chamber, the desalting chamber comprising a microfluidic channel, the microfluidic channel comprising a fluid inlet and a fluid outlet downstream the fluid inlet, wherein a valve is adjacent the fluid inlet; a piston disposed within the fluidic channel and proximate the fluid inlet; a force generator adjacent to the piston; and a porous material within the fluidic channel, wherein the porous material is disposed between the valve the fluid outlet; providing a signal to the force generator to move the piston; flowing the sample through the porous material into the fluid outlet; flowing the sample into a sensing chamber, wherein the sensing chamber and the desalting chamber are fluidically connected via the microfluidic channel; and sensing an analyte within the sample.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIG. 1A is a schematic diagram of a system for electrophoretic desalting, according to some embodiments;

FIG. 1B is a schematic illustration of the desalting chamber for capacitive desalting where two electrodes, biased at positive and negative voltages, attract ions of opposite polarity, and a nanoporous resin sits on the electrodes, which traps the small salt ions but leaves larger analyte particles in solution, according to one set of embodiments

FIGS. 1C-1D are schematic diagrams of a piston-driven desalting system, according to some embodiments;

FIGS. 1E-1F are schematic diagrams of a bimetallic strip as the force generator of the piston with a resistive heater configured to activate the bimetallic strip, according to some embodiments;

FIGS. 1G-1H depict schematic illustrations of a loaded spring as the force generator of the piston configured with an activating switch, according to one set of embodiments;

FIGS. 1I-1J are schematic illustrations of a compressed air canister as the force generator configured with an activating switch, according to some embodiments;

FIGS. 1K-1L show schematic illustrations of a piezoelectric tube as the force generator and an activating switch, according to some embodiments;

FIGS. 1M-1N are schematic diagrams of coiled shape memory alloy as the force generator configured with a resistive heater to activate the force generator, according to one set of embodiments;

FIG. 2 is a schematic showing how a desalting module can be placed upstream from a sensor module in the same microfluidic framework, allowing only desalted solution to be sensed, according to some embodiments;

FIG. 3A is schematic depiction of analytes proximate a sensor when the sample has been desalted, according to some embodiments; and

FIG. 3B is a schematic depiction showing the necessity of desalting where charged analytes with no surrounding ions create a measurable field, but in a solution with ions, the field beyond the Debye length is significantly reduced and precision charge-based detection of the charged analyte, such as with a FET, is significantly reduced, according to one set of embodiments.

### DETAILED DESCRIPTION

Charge-based biosensors of analytes in a sample (e.g., ionic fluids) can suffer from reduced sensitivity due to Debye screening of charges on the analyte by undesired ions. As described herein, this problem can be overcome through removal of free ions in the vicinity of a sensor, as is appreciated and recognized by the inventors. In some embodiments, such a sample requires adding a desalting region upstream from the sensor. In some embodiments, the sample fluid is first sent through the desalting region, where the ions are removed, then to the sensor region, where analytes are detected. The final reduced ion concentration increases the Debye length of the analyte molecules, increasing charge-based sensor sensitivity.

In some embodiments, methods for efficiently removing free charges (e.g., ions) in a sample, such as a biological sample, are described in order to increase sensitivity of field-effect biosensors. Such biosensors are used to determine the presence of analytes (e.g., biomolecules) including but not limited to proteins, protein fragments, DNA fragments, viruses, enzymes, and disease markers. Field-effect biosensors, such as a Si nanowire field-effect transistor (FET) sensors, measure properties that depend on the charge of the analyte for detection.

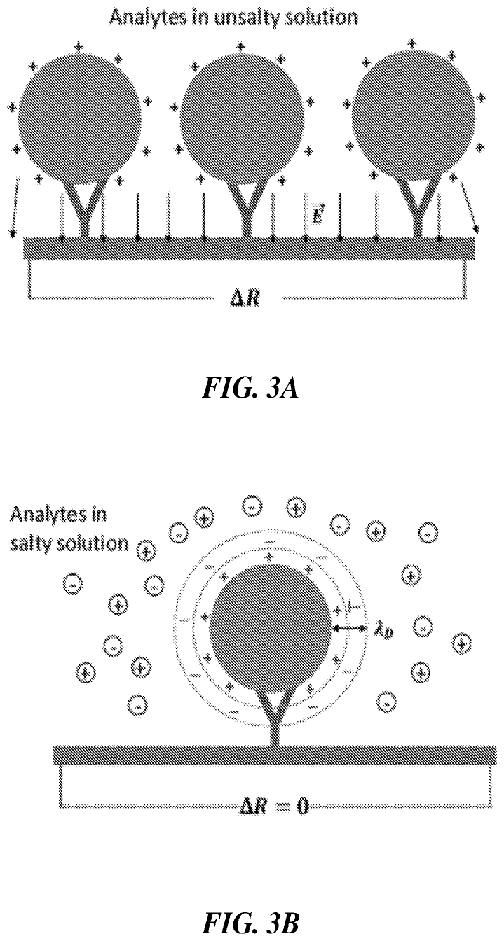

In some embodiments, the measured sensor property is the conductivity of a semiconducting channel. In such an embodiment, antibodies or other biomolecule-specific binding sites such as DNA (which can be used as the detector) are attached to the surface of a semiconducting nanowire. In some embodiments, the nanowire is made from silicon, germanium, or a III-V semiconductor. In some embodiments, the nanowire is a carbon nanotube. When the specific analyte (e.g., a biomolecule) binds to the detector, it is held close to the nanowire for a period of time, as shown in FIG. 3A. The charge on the analyte creates an electric field, which gates the semiconductor channel and changes its conductivity, as illustrated schematically in FIG. 3A for a charged analyte in free space (outside of the ion-rich solution). In some embodiments, a measured resistance (or conductance) change, ΔR, indicates the presence of the analyte. Without wishing to be bound by any theory, this may be the same phenomenon used in a metal-oxide-semiconductor FET (MOSFET), where an external gate voltage is applied to turn the semiconductor from insulating to conducting. In certain embodiments, charge is detected through a change in the surface plasmon resonance. However, some embodiments can use a different charge detection method.

In some cases, the analyte of interest is suspended in a biological fluid sample, including but not limited to, blood, sweat, or lacrimal fluid. Such fluids, in their natural state, contain not only analytes, but also other large and small molecules. In many cases, the fluids contain a high concentration of free charged atoms or small molecules (i.e., ions) including but not limited to Na⁺, K⁺, Cl⁻, Ca²⁺, Mg²⁺, CO³⁻. The presence of such example ions in a sample can hinder charge-based analyte detection, as described below, which greatly reduces sensitivity to subthreshold values. Removing the ions from (i.e., desalting) the sample can therefore increase the sensitivity to the charge of the analytes. Some sensors take samples that have already been desalted. In these cases, desalting occurs external to the sensor. The inventors have recognized and appreciated that a small sample can be used for analyte detection, and the entire process can be contained in a small device, where existing systems and methods may require larger, bulky centrifuges or may require proper alignment with gravitational forces provide for slow desalting. Systems and methods described herein and appreciated and recognized by the inventors may use electrophoretic or piston-assisted desalting such that detection can occur in a timely fashion.

Systems and methods described herein may be useful in desalting a sample containing an analyte prior to sensing of the analyte. As described herein, desalting refers to the removal of at least some of a plurality of ions within the sample that are not the analyte. For example, if a sample comprises a target analyte a plurality of sodium and chloride ions that are not the analyte, then desalting the sample will remove at least a portion (or all) of the sodium and chloride ions and the target analyte can be detected with less background compared to if the target analyte was detected with the sodium and chloride ions still present in the sample. In some embodiments, the analyte may also be an ion, and those skilled in the art will understand that the analyte will not be removed by desalting, but rather only non-analyte ions will be removed. In some embodiments, the analyte has a larger mass than any one ion of the plurality of ions such that the analyte may be selected by size, while the undesired ions are removed by the sample by desalting.

As used herein, “salts” are given their ordinary meaning to refer to compounds comprised of ions, i.e., cations and/or anions. As mentioned above, non-limiting examples of salts include Na⁺, K⁺, Cl⁻, Ca²⁺, Mg²⁺, CO³⁻. However, certain biological molecules, such as amino acids, proteins, nucleic acids, DNA, carbohydrates, and others can be salts, as the term used herein is not so limiting. Those of ordinary skill in the art are capable of selecting an analyte (e.g., a biomolecule) to be sensed and to screen unwanted ions by desalting regardless of if the ions are atomic ions (e.g., Na⁺, Cl⁻) or biomolecular ions (e.g., an amino acid). The selection of analyte and ions to be removed can be made, as one example, by the choice of porous material or resin used during desalting. Other methods of selection as possible, such as by applying an appropriate voltage to an electrode within the system.

Some embodiments may contain a porous material. “Porous material” is given its ordinary meaning in the art as a material that contains pores. These pores may also be gaps, voids, or channels, and can be continuous or non-continuous. In some embodiments, the porous material is a microporous material. In some embodiments, the porous material is a nanoporous material. The porous material may comprise any suitable material for removing ions from the sample. Non-limiting examples of suitable porous materials include graphene, molybdenum disulfide (MoS₂), polyaniline nanofibers, cellulose nanofibers, copolymer membranes, organosilica membranes, carbon nanofibers, carbon nanotubes, gelatin nanoporous membranes, zeolite nanoporous membranes, and block copolymer-based membranes. In some embodiments, the porous material comprises a microporous or mesoporous material, such as a microporous or mesoporous silica-based membranes, and microporous or mesoporous inorganic membranes (e.g., metal-based membranes, ceramic based membranes, oxide-based membranes), as non-limiting examples. For some embodiments, the porous material can be an engineered structure, which are membranes or porous structures fabricated used top-down or bottom-up lithography or other lithographical growth processes. In such embodiments, the pore size (e.g., an average pore diameter) can be 1000 μm-1 nm.

In some embodiments, the porous material comprises a microporous material (e.g., a microporous resin). In some embodiments, the microporous material has an average pore diameter no greater than 1000 microns, no greater than 800 microns, no greater than 600 microns, no greater than 400 microns, no greater than 200 microns, no greater than 100 microns, no greater than 50 microns, no greater than 10 microns or no greater than 1 micron. In some embodiments, the microporous material has an average pore diameter of at least 1 micron, at least 10 microns, at least 50 microns, at least 100 microns, at least 200 microns, at least 400 microns, at least 600 microns, at least 800 microns, or at least 1000 microns. Combinations of the above-referenced ranges are also possible (e.g., at least 10 microns and no greater than 100 microns). Other ranges are possible.

In some embodiments, the porous material comprises a nanoporous material (e.g., a nanoporous resin). In some embodiments, the nanoporous material has an average pore diameter no greater than 1000 nanometers, no greater than 800 nanometers, no greater than 600 nanometers, no greater than 400 nanometers, no greater than 200 nanometers, no greater than 100 nanometers, no greater than 50 nanometers, no greater than 10 nanometers or no greater than 1 nanometer. In some embodiments, the microporous material has an average pore diameter of at least 1 nanometer, at least 10 nanometers, at least 50 nanometers, at least 100 nanometers, at least 200 nanometers, at least 400 nanometers, at least 600 nanometers, at least 800 nanometers, or at least 1000 nanometers. Combinations of the above-referenced ranges are also possible (e.g., at least 10 nanometers and no greater than 100 nanometers). Other ranges are possible.

Embodiments described herein are general to detection based purely on analyte charge and covers embodiments that involve detection based on the analyte's charge properties. This includes the above-mentioned field-effect transistors, but may also include other sensor designs and methods.

### Description of the Concept

A charged particle (e.g., a nanoparticle) in an ion-rich fluid, such as blood, will attract local opposite charges (generally smaller, mobile species such as Na⁺, K⁺, Cl⁻, Ca²⁺ and other ions), as illustrated in FIG. 3B. The net result is an electrically neutral body consisting of a charged dielectric interior and an oppositely charged surface shell, as illustrated in FIG. 3B. The charged shell thickness can be given by the Debye length λ_{D}, which depends on the ionic valences z and concentrations n within the fluid, the fluid's dielectric constant E, and the temperature T,

$${λ}_{D} = \sqrt{\frac{{ϵϵ}_{0} {k}_{B} T}{{e}^{2} {\sum }_{i} {n}_{i} {z}_{i}^{2}}} \qquad Equation 1$$

Here, i refers to each distinct ionic species in the fluid. From far away the particle looks neutral, and therefore cannot be detected electrically. Very close to the particle, within one Debye length of the particle's surface, the particle can be detected by its electric field. Outside of the Debye length, the particle's charge is fully screened so that the measurable total electric field is zero. The Debye length for a nanoparticle in a fluid such as blood is typically of order 1-10 nm. In some embodiments, the intrinsic charge on the nanoparticle is in its interior. In some embodiments, the charge is on the nanoparticle's surface. In some embodiments, the charge is evenly distributed. In certain embodiments, the charge is localized. The approaches described herein are general and covers many possible charge distribution possibilities for the nanoparticle.

Some embodiments describe field-effect transistor nanowires. However, the principles are general, and one skilled in the art can easily extend the systems and methods described herein to other charge-sensitive detection methods.

If the charged analyte is in solution, and is within the Debye length of the nanowire surface, its electric field can act to gate the nanowire. However, most detectors (such as antibodies) that are functionalized onto the nanowire are ˜10 nm in length, of the same order as or greater than the Debye length, as shown in FIGS. 3A-3B. In some embodiments, the Debye length is less than 1 nm. In some embodiments, the Debye length is 1-10 nm. In certain embodiments, the Debye length is greater than 10 nm. The sensitivity to the analyte (e.g., a protein) is then very limited, or binding may even be undetectable, as the analyte will appear to be electrically neutral and produce no electric field at the location of the nanowire sensor. It is therefore of great importance to be able to modify the Debye length of an analyte (e.g., nanoparticles) in ion-rich fluids such that the nanoparticles can be detected electrically.

In some embodiments, a method of increasing bio sensor performance through increasing the nanoparticle Debye length by decreasing the ionic concentration in the vicinity of the sensor is described. By Eq. 1, the Debye length is inversely proportional to the inverse square root of the ionic concentration. A reduction of the ionic concentration can therefore lead to an increase in the Debye length, and an increase in the nanoparticle's electric field at the sensor substrate. Removal of the ions (i.e., “desalting”) therefore can enable analyte detection that otherwise would not have occurred.

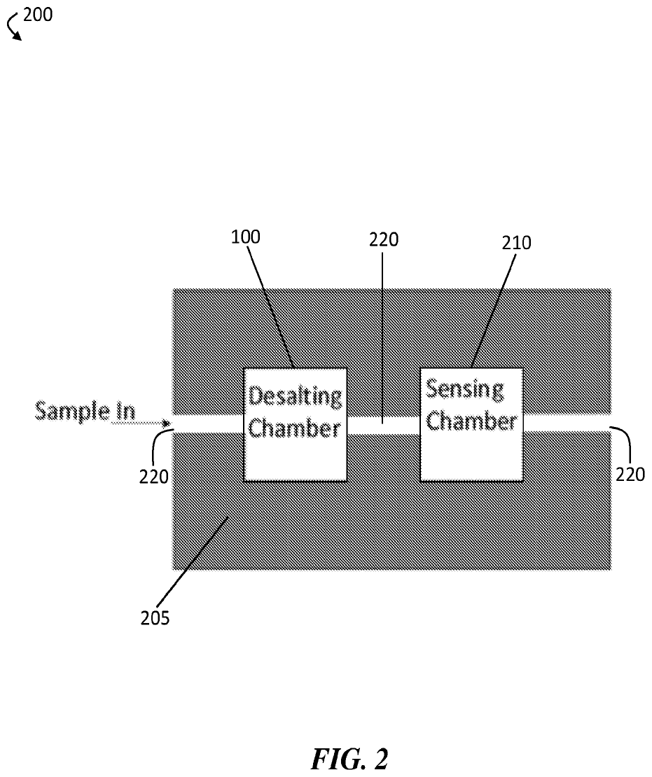

Systems described herein can integrate the desalting module with the biosensor module within one microfluidic framework. In some embodiments, the desalting is accomplished upstream from the biosensing, as in FIG. 2. The sample first enters the desalting region 100 through microfluidic channel 220, where the ions are removed, then enters the sensor region 210, where the presence of analytes is determined. In some embodiments, the desalting chamber and sensor chamber are on the same substrate. In some embodiments, the desalting chamber and sensing chamber are on different substrates. In some embodiments the chambers are connected via a microfluidic channel. In some embodiments, the single microfluidic structure enables direct desalting, without the need for centrifuges, alignment with the force of gravity, other equipment, or sample transfer between devices. An advantage over current existing systems for desalting is that the microfluidic nature of this invention can also enable the direct desalting in lab-on-a-chip scale, and enables device miniaturization to the point where disposable or implantable sensors are possible.

In some embodiments, the chambers are arranged horizontally. In certain embodiments, the chambers are arranged vertically. However other geometries for arranging the two chambers are possible, provided that such that desalting occurs before the final measurement.

In some embodiments, the ion concentration is measured prior to and/or after desalting. This gives a baseline for which analyte concentration can accurately be determined. In certain embodiments, the measurement of ion concentration is used as an additional indicator for the presence of certain analytes.

Electrophoretic Separation for Desalting

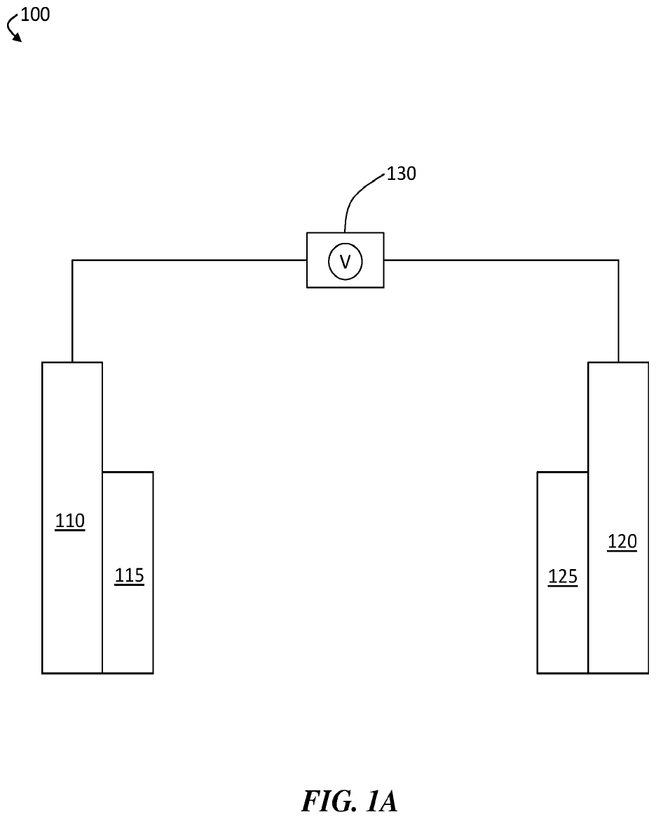

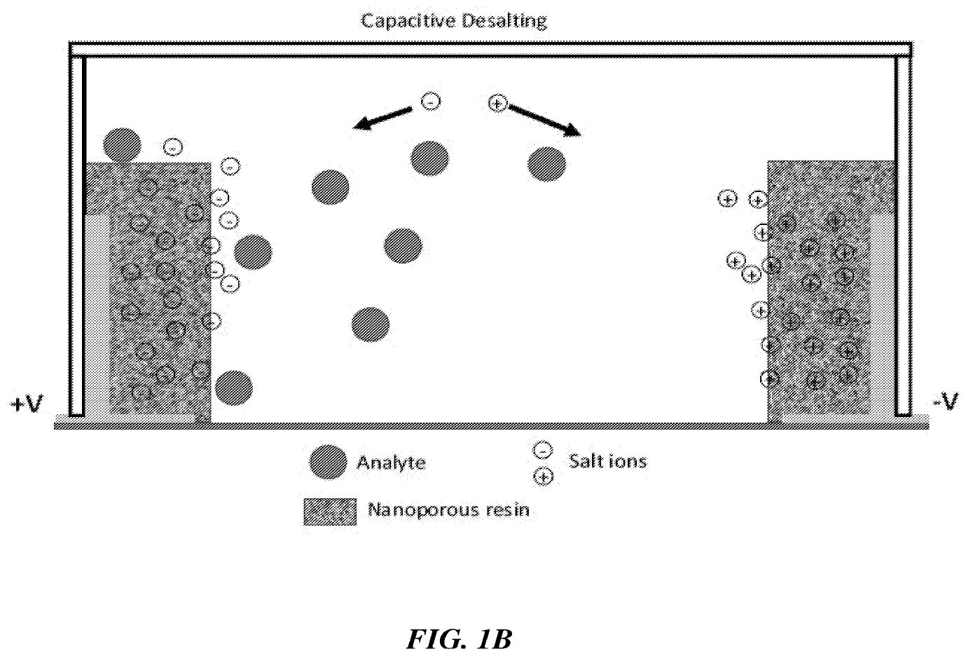

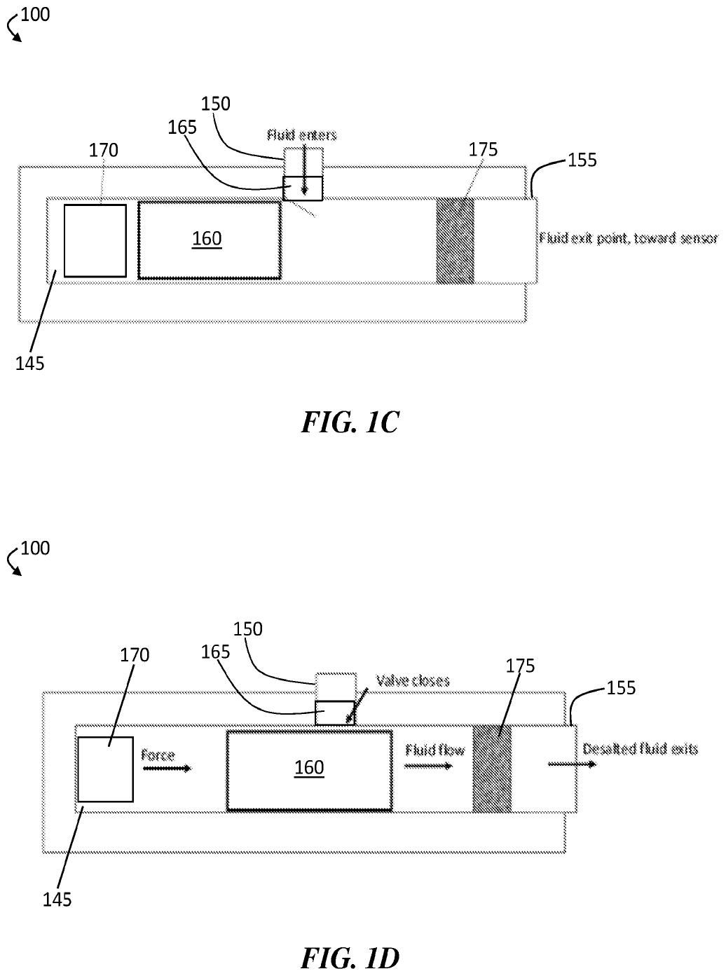

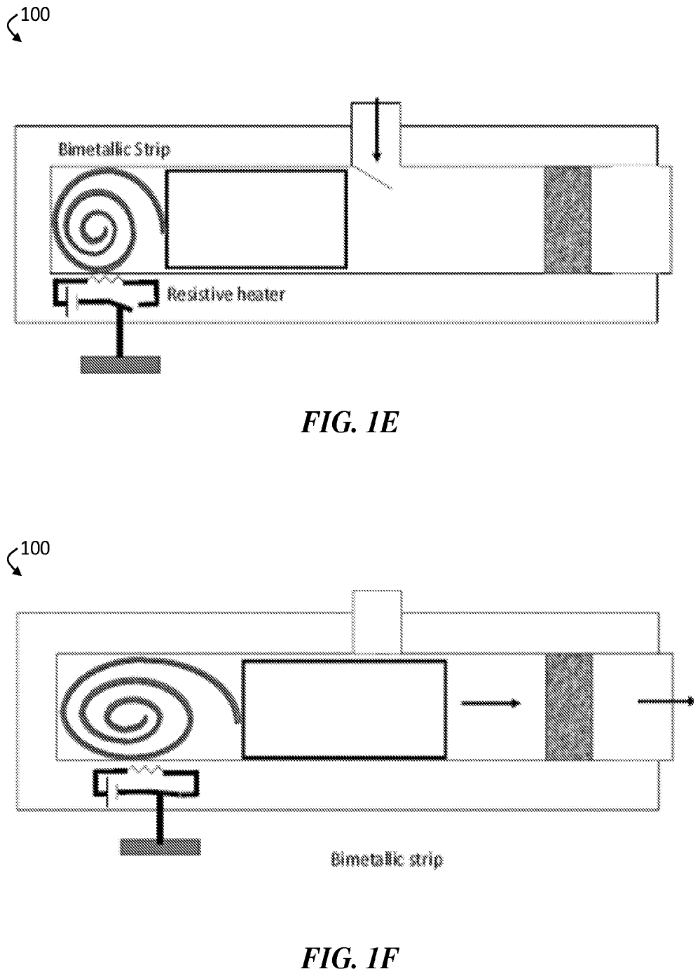

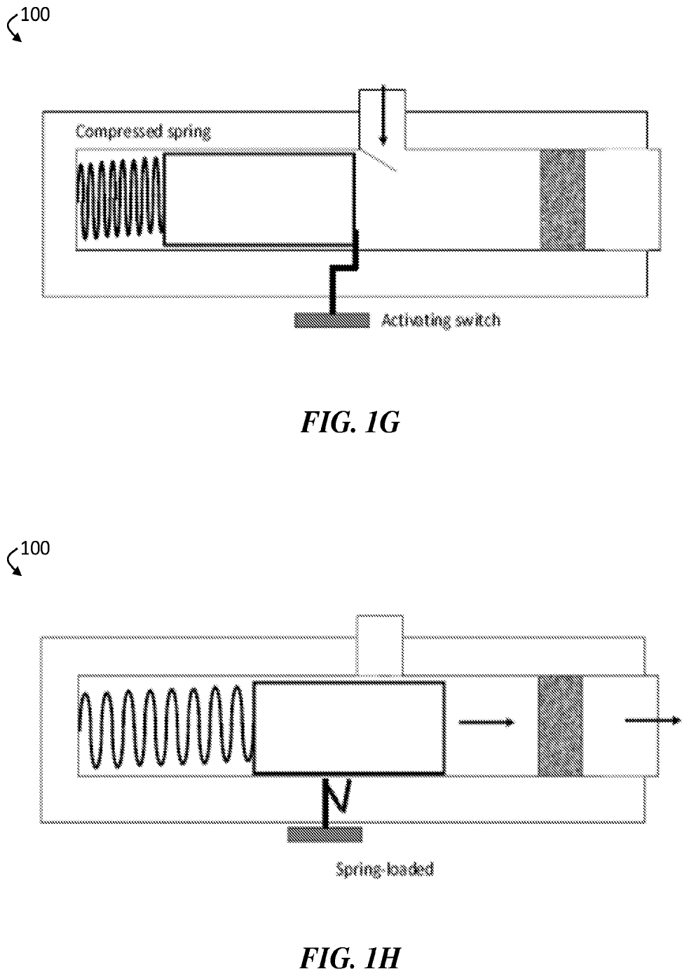

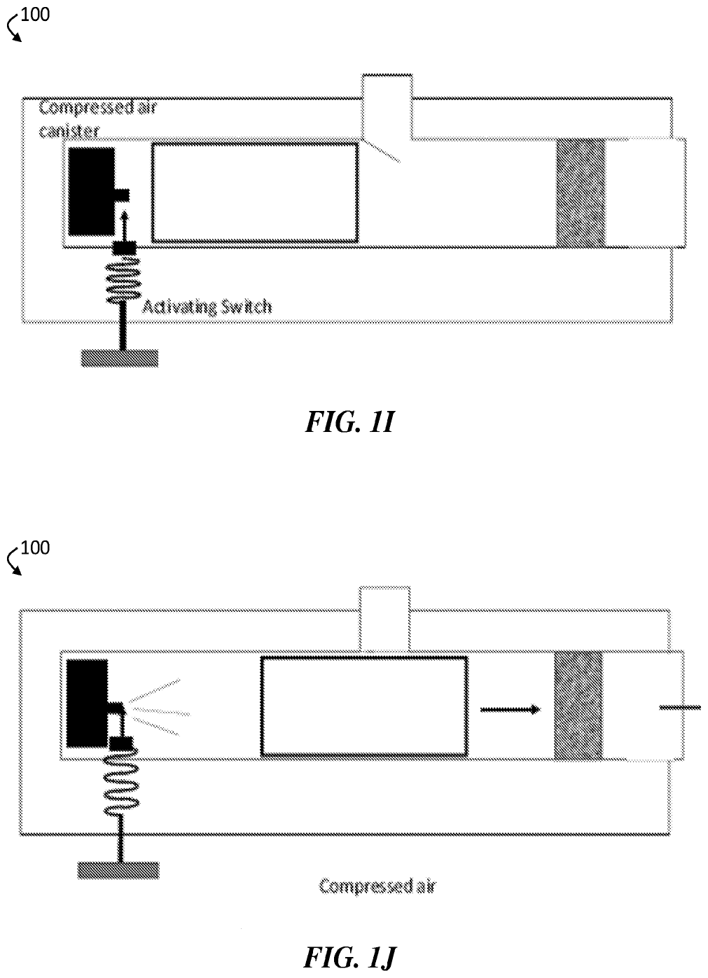

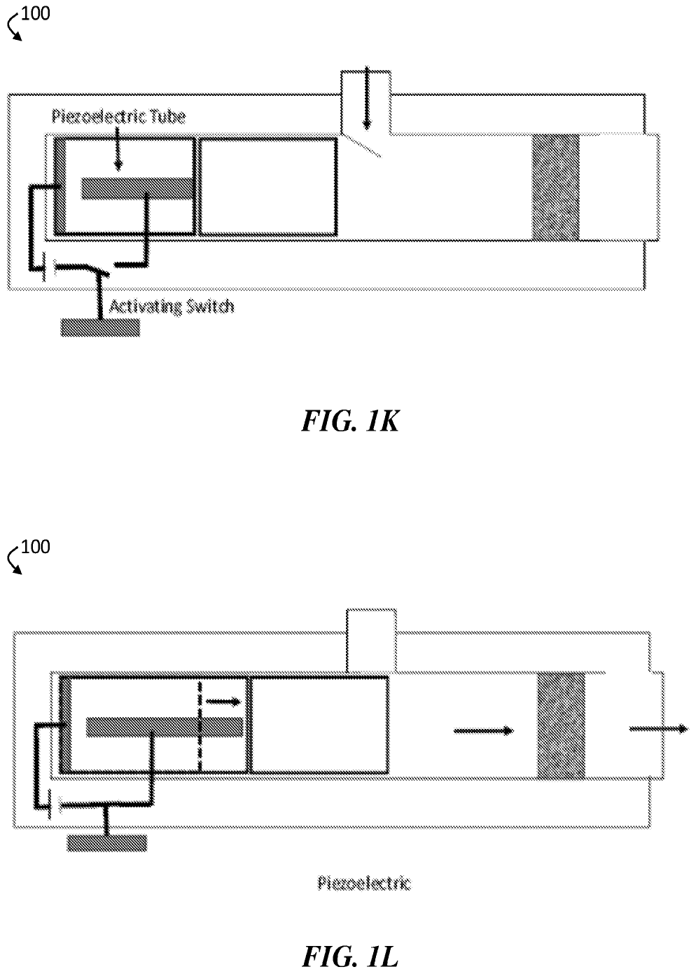

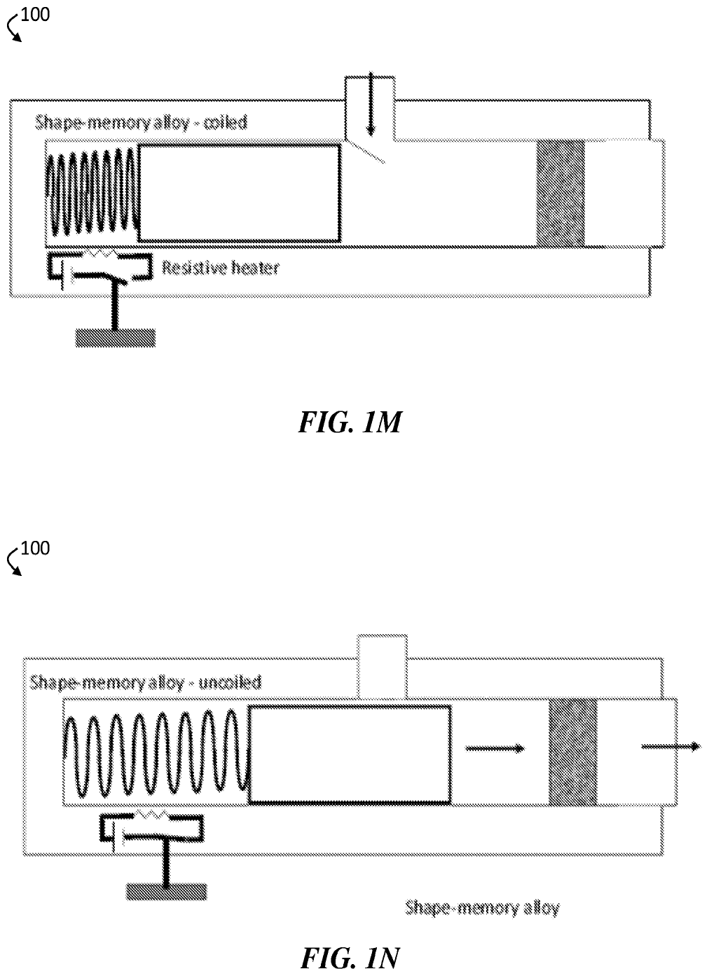

In accordance with some embodiments, capacitive electrophoresis is used to remove salt from solution as it flows through a microfluidic channel. FIG. 1A shows a schematic of the electrophoretic separation concept. A single microfluidic channel is needed, which contains two electrodes, a first electrode 110 and a second electrode 120. One electrode can be biased at positive voltage, the another at negative voltage. Such voltages may be provided by, for example, a potentiostat 130, which can place first electrode 110 and second electrode 120 in electric communication with one and other. The electrical nature of this invention allows it to be miniaturized, as it does not require centrifuges or other external equipment.

The positive electrode attracts the negative charges, while the negative electrode attracts the positive charges. Because the small salt ions are significantly more mobile than the larger analyte molecules, the salt ions are initially attracted to the electrodes and rapidly diffuse toward the electrodes, leaving the charged analytes to more slowly migrate. This process is schematically illustrated in FIG. 1B. Equilibrium is reached when enough salt reaches the electrodes to cancel out the applied voltage. In general, the amount of desalting is controllable with the applied voltage.

The surface charge density accumulating at any surface can be estimated using the Gouy-Chapman equation,

$$σ = 2 \sqrt{\frac{2 {RTC}_{0}}{{ϵϵ}_{0}}} sinh ( \frac{z F ψ ( 0 )}{2 R T} ) ,$$

where σ is the surface areal charge density, ∈∈₀ is the dielectric constant of the fluid, R is the gas constant, F is the Faraday constant, C₀ is the initial bulk salt concentration, z is the ionic number of the salt ions, and ψ(0) is the potential applied to the electrode. The voltage applied to the electrode needs to be large enough such that the total charge accumulating at the electrode surface is equal to the amount of charge that needs to be removed from the fluid sample. In certain embodiments, this is accomplished by increasing the surface area of the electrodes, including utilizing metal nanopillars, nanospheres, corrugation, or other topographical features. The electrode area must be large enough to absorb all the ions (e.g., the salt). In some embodiments, the effective electrode area is enhanced through surface roughening. In some embodiments, nanopillars or nanowire nests are used to enhance the area. In some embodiments, the area is enhanced by making the channel very long. Our inventions cover all such methods of extending the area to maximize desalting potential.

In certain embodiments, the electrodes are coated with a nanoporous material, such as a first nanoporous material 115 and a second nanoporous material 125 in FIG. 1A, that is selective to the smaller salt ions but not to the larger analyte molecules. The nanoporous material could be an oxide, a polymer, a resin, or a collection of nanoparticles. Other materials with and without size-segregation capabilities can be used. In some embodiments, the material contains dangling bonds to which ions can attach. In such embodiments, the ions are captured in the material and will not diffuse back into solution. In certain embodiments, the resin does not contain dangling bonds, and the salt can diffuse back into solution. In such embodiments, the fluid flow rate and voltage timescales are crucial to performance.

In certain embodiments, the voltage is pulsed. The pulse width and rate are chosen so that the ions react but the analyte particles do not. For such embodiments, the salt ions must be trapped with the nanoporous resin to prevent diffusion back into the fluid sample.

In certain embodiments, the fluid sample is flowed fast enough through the channel so that the salt is all removed by the electrodes while the analytes flow through.

### Forced Flow Microporous Resin Desalting

Pertinent to some embodiments, a piston forces the fluid through a microporous resin, which removes the salt (e.g., ions). The microporous resin can be similar to that found in commercially available gravitational or centrifugal desalting columns. Different methods exist for providing the force (i.e., a force generator) needed to drive the piston. The force can be actuated through any number of means, including a user activated switch, or an automatic relay. Other means for activating the piston are possible.

In some embodiments, the sample fluid enters a chamber with microporous resin at one end and a piston at the other, as in FIG. 1C-1D. For example, a microfluidic channel 145 comprises fluid inlet 150 through which the sample can enter the microfluidic channel. In some embodiments, when the chamber is full, a microfluidic valve, such as valve 165, closes so that the sample does not escape. The piston 160 is then actuated by force generator 170, which drives the fluid through the desalting membrane 175 and towards the sensor through fluid outlet 155. The following discusses a number of embodiments for actuating the piston.

Bimetallic strip embodiments: Here, a standard microfluidic desalting column is used. This consists of the fully enclosed microfluidic channel with desalting resin at one end, schematically depicted in FIG. 1E-1F. Beyond the desalting resin the microfluidic channel extends to the entry point of the sensing chamber. At the other end of the microfluidic channel is a piston. Just at the edge of the piston is a connecting inlet from which the sample enters the channel. In some embodiments, after the sample enters, a door will close on the entry point to seal the channel. Beyond the piston is a coiled bimetallic strip. When the sample is to be desalted, a switch is released, which supplies a current to a heater. The heat causes the materials in the bimetallic strip to expand. The two materials in the bimetallic strip have different thermal expansion coefficients, which causes the coil to unravel as it is heated. The uncoiling provides a mechanical force on the piston. The piston is reset by allowing the bimetallic strip to cool.

In some embodiments, the heating is initiated by the user by pressing a switch or button situated on the outside packaging of the device.

According to certain embodiments, the heating is initiated automatically. A sensor inside the device determines that enough sample fluid has entered, and this triggers an electrical signal that releases the switch to the heater. Other user and automatic initiation of the signal are also contemplated.

Compressed Air Forcing embodiments: Here, a standard microfluidic desalting column is used. This consists of the fully enclosed microfluidic channel with desalting resin at one end. Beyond the desalting resin the microfluidic channel extends to the entry point of the sensing chamber. At the other end of the microfluidic channel is a piston. Just at the edge of the piston is a connecting inlet from which the sample enters the channel. In some embodiments, after the sample enters, a door will close on the entry point to seal the channel. Beyond the piston is a chamber containing a compressed gas cartridge. In some embodiments, the chamber is filled with fluid. In certain embodiments, the chamber is filled with an inert gas. When the sample is to be desalted, the compressed air cartridge is opened, schematically illustrated in FIGS. 1I-1J. The escaping gas pushes on the piston, which forces the sample fluid through the desalting resin and into the sensing chamber.

In some embodiments, the compressed air cartridge is opened by puncturing with a needle. In certain embodiments, a pull-tab opens the compressed air cartridge. Our invention covers all methods of releasing the compressed air from the cartridge.

In some embodiments, the opening of the compressed air cartridge is initiated by the user. In such embodiments, a button on the outside of the device packaging is pressed, switch is thrown, or some other simple user interface is employed. This action begins the chain of events that releases the compressed air. In some embodiments, a spring-loaded needle is released through the user interface, which punctures the compressed air cartridge. In certain embodiments, the user interface triggers an electrical signal, which leads to the compressed air cartridge opening. This invention covers all possible user-triggered mechanisms.

According to certain embodiments, the compressed air cartridge is initiated automatically. A sensor inside the device determines that enough sample fluid has entered, and this triggers an electrical signal that initiates the compressed air cartridge deployment.

Spring-Loaded Forcing embodiments: Here, a standard microfluidic desalting column is used. This consists of the fully enclosed microfluidic channel with desalting resin at one end. Beyond the desalting resin the microfluidic channel extends to the entry point of the sensing chamber. At the other end of the microfluidic channel is a piston. Just at the edge of the piston is a connecting inlet from which the sample enters the channel. In some embodiments, after the sample enters, a door will close on the entry point to seal the channel. Beyond the piston is a chamber containing a compressed spring, schematically illustrated in FIGS. 1G-1H. The piston is held in place with a mechanical switch, keeping the spring compressed. When the sample is to be desalted, the switch is released, allowing the piston to move. The spring pushes on the piston, which forces the sample fluid through the desalting resin and into the sensing chamber.

In some embodiments, the switch release is initiated by the user. In such embodiments, a button on the outside of the device packaging is pressed, or a switch repositioned, causing the internal switch to be released.

According to certain embodiments, the spring release is initiated automatically. A sensor inside the device determines that enough sample fluid has entered, and this triggers an electrical signal that releases the mechanical switch holding the piston. In some embodiments, the spring mechanism is integrated with a spring that initiates blood sampling.

Piezoelectric Forcing embodiments: Here, a standard microfluidic desalting column is used. This consists of the fully enclosed microfluidic channel with desalting resin at one end. Beyond the desalting resin the microfluidic channel extends to the entry point of the sensing chamber. At the other end of the microfluidic channel is a piston. Just at the edge of the piston is a connecting inlet from which the sample enters the channel. In some embodiments, after the sample enters, a door will close on the entry point to seal the channel. Beyond the piston is a piezoelectric tube, schematically illustrated in FIGS. 1K-1L. When the sample is to be desalted, a switch is released, applying a voltage to the piezoelectric tube (i.e., piezotube). The piezoelectric tube expands with the applied voltage, creating a force on the piston.

In some embodiments, a piezoelectric motor is used. In such embodiments, the piezoetube uses “inchworm” motion to press the piston. According to certain embodiments, the expansion of the piezotube directly forces the piston.

In some embodiments, the switch release is initiated by the user. In such embodiments, a button on the outside of the device packaging is pressed, or a switch repositioned, causing the internal switch to be released.

According to certain embodiments, the switch to the piezotube is initiated automatically. A sensor inside the device determines that enough sample fluid has entered, and this triggers an electrical signal that releases the mechanical switch holding the piston.

Shape-memory alloy embodiment: Here, a standard microfluidic desalting column is used. This consists of the fully enclosed microfluidic channel with desalting resin at one end. Beyond the desalting resin the microfluidic channel extends to the entry point of the sensing chamber. At the other end of the microfluidic channel is a piston. Just at the edge of the piston is a connecting inlet from which the sample enters the channel. In some embodiments, after the sample enters, a door will close on the entry point to seal the channel. Beyond the piston is a shape-memory alloy, schematically illustrated in FIGS. 1M-1N. When the sample is to be desalted, a switch is released, which supplies a current to a heater. The heater changes the temperature of the shape-memory alloy in such a way that it applies a force to the piston. The piston is reset by cooling the shape-memory alloy, which returns to its initial state.

In some embodiments, the heating is initiated by the user by pressing a switch or button situated on the outside packaging of the device.

According to certain embodiments, the heating is initiated automatically. A sensor inside the device determines that enough sample fluid has entered, and this triggers an electrical signal that releases the switch to the heater.

While the above embodiments describe several force generators (e.g., a bimetallic strip, compressed air forcing, a spring, a piezoelectric, a shape memory alloy), those of ordinary skill in the art understand that other force-generating devices may be used so long as the force-generating device provides adequate force to the piston to move a sample to a downstream position and through the porous material to achieve desalting.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law. As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Some embodiments may be embodied as a method, of which various examples have been described. The acts performed as part of the methods may be ordered in any suitable way. Accordingly, embodiments may be constructed in which acts are performed in an order different than illustrated, which may include different (e.g., more or less) acts than those that are described, and/or that may involve performing some acts simultaneously, even though the acts are shown as being performed sequentially in the embodiments specifically described above.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A system for removing a plurality of ions from a sample, the system comprising:
a first electrode;
a first porous material adjacent to at least a portion of the first electrode;
a second electrode in electrical communication with the first electrode; and
a second porous material adjacent to at least a portion of the second electrode.

2. A system for removing a plurality of ions from a sample, the system comprising:
a substrate;
a desalting chamber proximate the substrate, the desalting chamber comprising:
a first electrode,
a first porous material adjacent to at least a portion of the first electrode,
a second electrode in electrical communication with the first electrode, and
a second porous material adjacent to at least a portion of the second electrode;
a microfluidic channel; and
a sensing chamber proximate the substrate, the sensing chamber comprising:
at least one sensor,
wherein the desalting chamber and the sensing chamber are in fluidic communication via the microfluidic channel.

3. A method of removing a plurality of ions from a sample, the method comprising:
flowing the sample into a desalting chamber, the desalting chamber comprising:
a first electrode,
a first porous material adjacent to at least a portion of the first electrode,
a second electrode in electrical communication with the first electrode, and
a second porous material adjacent to at least a portion of the second electrode;
applying a first voltage of a first sign to the first electrode;
applying a second voltage of a second sign to the second electrode;
attracting at least a portion of the plurality of ions towards the first electrode and the second electrode;
flowing the sample into a microfluidic channel;
flowing the sample into a sensing chamber, wherein the sensing chamber and the desalting chamber are fluidically connected via the microfluidic channel; and
sensing an analyte within the sample.

4. The system of claim 2, wherein the desalting chamber is positioned adjacent the substrate.

5. The system of claim 2, wherein the desalting chamber and the sensing chamber are positioned adjacent the substrate.

6. The system of claim 2, further comprising a second substrate, wherein the sensing chamber is positioned adjacent the second substrate.

7. The system or method of any one claims 1-6, wherein the sensor comprises a field effect biosensor.

8. The system or method of any one of claims 1-7, wherein the sensor comprises a silicon nanowire and at least one antibody.

9. The system or method of any one of claims 1-8, wherein the sensor is configured to measure the conductivity and/or the resistance of an analyte attached to the sensor.

10. The system or method of any one of claims 1-9, wherein the porous material comprises an oxide, a polymer, a resin and/or a plurality of nanoparticles.

11. The system or method of any one of claims 1-10, wherein the porous material comprises a size-exclusion material.

12. The system or method of any one of claims 1-11, wherein the porous material comprises dangling bonds configured to associate with at least a portion of the plurality of ions.

13. The system or method of any one of claims 1-12, wherein the porous material comprises a nanoporous material.

14. The method of claim 3, comprising trapping at least a portion of the plurality of ions within the first porous material and/or the second porous material.

15. The method of any one of claim 3 or 14, wherein the analyte is larger than each ion of the plurality of ions.

16. The method of any one of claim 3 or 14-15, wherein applying the first voltage and/or applying the second voltage comprises a pulsed voltage, the pulsed voltage comprising a pulse width and a pulse rate.

17. The method of any one of claim 3 or 14-16, comprising sensing prior to and/or after any one of the flowing steps.

18. The method of any one of claim 3 or 14-17, wherein any one of the flowing steps comprises a first flow rate, a second flow rate, and/or a third flow rate.

19. A system for removing a plurality of ions from a sample, the system comprising:
a microfluidic channel, the microfluidic channel comprising:
a fluid inlet and a fluid outlet downstream the fluid inlet, wherein a valve is adjacent the fluid inlet;
a piston disposed within the fluidic channel and proximate the fluid inlet;
a force generator adjacent to the piston; and
a porous material within the fluidic channel, wherein the porous material is disposed between the valve the fluid outlet, and
wherein the piston is configured to move a sample downstream the microfluidic channel.

20. A system for removing a plurality of ions from a sample, the system comprising:
a substrate;
a desalting chamber proximate the substrate, the desalting chamber comprising:
a microfluidic channel, the microfluidic channel comprising:
a fluid inlet and a fluid outlet downstream the fluid inlet, wherein
a valve is adjacent the fluid inlet;
a piston disposed within the fluidic channel and proximate the fluid inlet;
a force generator adjacent to the piston; and
a porous material within the fluidic channel, wherein the porous material is disposed between the valve the fluid outlet; and
a sensing chamber proximate the substrate, the sensing chamber comprising:
at least one sensor,
wherein the piston is configured to move a sample downstream within the microfluidic channel, and
wherein the desalting chamber and the sensing chamber are in fluidic communication via the microfluidic channel.

21. A method of removing a plurality of ion from a sample, the method comprising:
flowing a sample into a desalting chamber, the desalting chamber comprising:
a microfluidic channel, the microfluidic channel comprising:
a fluid inlet and a fluid outlet downstream the fluid inlet, wherein
a valve is adjacent the fluid inlet;
a piston disposed within the fluidic channel and proximate the fluid inlet;
a force generator adjacent to the piston; and
a porous material within the fluidic channel, wherein the porous material is disposed between the valve the fluid outlet;
providing a signal to the force generator to move the piston;
flowing the sample through the porous material into the fluid outlet;
flowing the sample into a sensing chamber, wherein the sensing chamber and the desalting chamber are fluidically connected via the microfluidic channel; and
sensing an analyte within the sample.

22. The system of claim 20, wherein the desalting chamber is positioned adjacent the substrate.

23. The system of claim 20, wherein the desalting chamber and the sensing chamber are positioned adjacent the substrate.

24. The system of claim 20, further comprising a second substrate, wherein the sensing chamber is positioned adjacent the second substrate.

25. The system or method of any one of claims 19-24, wherein the fluid outlet of the microfluidic channel is arranged and adapt to provide fluidic communication to the sensing channel.

26. The system or method of any one of claims 19-25, wherein the sensor comprises a field effect biosensor.

27. The system or method of any one claims 19-26, wherein the sensor comprises a silicon microwire and at least one antibody.

28. The system or method of any one of claims 19-27, wherein the sensor is configured to measure the conductivity and/or the resistance of an analyte attached to the sensor.

29. The system or method of any one of claims 19-28, wherein the porous material comprises an oxide, a polymer, a resin and/or a plurality of nanoparticles.

30. The system or method of any one of claims 19-29, wherein the porous material comprises a size-exclusion material.

31. The system or method of any one of claims 19-30, wherein the force generator comprises a bimetallic switch, a compressed spring, a compressed air canister, a piezoelectric tube, and/or a shape-memory alloy coil.

32. The system or method of any one of claims 19-31, wherein the force generator comprises an activating switch and/or a resistive heater.

33. The system or method of any one of claims 19-32, wherein the porous material comprises a microporous material.

34. The system or method of any one of claims 19-33, wherein the porous material comprises a nanoporous material.

35. The method of claim 21, wherein the providing the signal step causes the any one of the flowing steps.

36. The method of any one of claim 21 or 35, comprising trapping at least a portion of the plurality of ions within the porous material.

37. The method of any one of claim 21 or 35-36, comprising sensing prior to and/or after the flowing through the porous material.

38. The method of any one of claim 21 or 35-37, wherein any one of the flowing steps comprises a first flow rate, a second flow rate, and/or a third flow rate.
